(19) United States
(12) Patent Application Publication
(10) Pub. No.: US 2010/0004522 A1
(43) Pub. Date: Jan. 7, 2010

(54) Continuously Wearable Compact Blood Glucose Measuring Device

(76) Inventor: Eddie Varela, Scottsville, NY (US)

Correspondence Address: LAW OFFICE OF TRACY P. JONG, 2775 BUFFALO ROAD, SUITE 3, ROCHESTER, NY, 14624, US

(21) Appl. No.: 12/495,037

(22) Filed: Jun. 30, 2009

Related U.S. Application Data
(60) Provisional application No. 61/077,489, filed on Jul. 2, 2008.

(51) Int. Cl. A61B 5/1468

(52) U.S. Cl. 600/347

(57) ABSTRACT

A wearable blood glucose measuring device based device and kit that incorporates non-continuous, invasive, electrochemical glucose test strip technologies for measuring blood glucose concentrations. The device can be inconspicuously worn due to its compact nature and integration into commonly worn articles such as watches, bracelets, armbands, wristbands, and the like. All-in-one embodiments include integrated test strip management including automatic test strip dispensing/ejection as well as incorporating spring loaded lancet apparatus capable of the dispensing, arming and activating functions enabling the user to provide a blood sample for glucose testing. Some embodiments do not integrate, nor contain any lancing devices/lancets. Other embodiments contain secure storage compartments integrated into the wearable blood glucose measuring device where the user can choose to store loose glucose test strips, calibration strips, lancets, cleaning wipes, and any combination thereof.

### CROSS REFERENCE TO RELATED APPLICATIONS AND PRIORITY CLAIM

This application claims priority to provisional application U.S. Ser. No. 61/077,489 filed on Jul. 2, 2008.

### BACKGROUND OF THE INVENTION

1. Field of the Invention

This invention relates generally to a wearable invasive blood glucose measuring or monitoring device system, and more particularly to a system that includes an all-in-one wearable kit embodiment utilizing blood glucose measuring technology using invasive, electrochemical, single use glucose test strips, where the system includes storage for necessary peripherals. The present invention incorporates well known, non-continuous, test strip based blood glucose measurement technologies. Additionally, disclosed is a wearable device capable of accepting various blood glucose measuring system components such as a separate glucose measuring device, test strips, lancets, and the like.

2. Description of Related Art

Blood glucose measuring kits come in diverse shapes and sizes; the kit contents as well as the features of the included contents vary from kit to kit. While these existing and previously known kits are presumably adequate for their intended purpose, none of these kits are configured adequately such that they can be inconspicuously worn. Additionally, these existing and previously known kits are not designed to be worn, nor assist the operator in the act of taking a blood glucose reading, but they merely provide storage for the individual components required for blood glucose testing.

U.S. Patent Application No 2006/0040333A1 to Zocchi entitled “Blood Glucose Monitoring Kit” explains:
Although useful in simplifying the handling of a large quantity of individual components, kits of this type suffer from a couple notable disadvantages.
As a first disadvantage, it has been found that kits of the type described above are somewhat bulky in size. In particular, the relatively large size of conventional blood glucose monitors tends to significantly increase the overall size (and, in particular, the thickness) of the kit. As a result, the patient often finds it to be considerably uncomfortable to store the kit on his/her person (e.g., in a clothing pocket) between tests, which is highly undesirable.
As a second disadvantage, the fact that all of the blood glucose testing components are removably stored within the case increases the number of preparatory steps that a patient must undertake prior to performing an assay.
Specifically, the user must first open the case (e.g., by unzipping, unsnapping, etc.) in order to access the various components contained therein. With the case open, the user must then remove the lancet base and blood glucose monitor therefrom. Preferably, the monitor is then positioned on a flat and stable surface to facilitate its use. Next, the patient must remove a disposable lancet and a disposable test strip from the pouch. In turn, the disposable lancet and the disposable test strip must be unwrapped and installed into their corresponding tools. Only after completion of all these preparatory steps can the user perform an assay, which is highly undesirable.

U.S. Pat. Application No. US 2005/0054907 A1 to Page, entitled “Highly Portable and Wearable Blood Analyte Measurement System” discloses a non-invasive system for continuous monitoring of blood glucose concentrations. This system uses a photoacoustic measurement scheme where laser pulses are used to stimulate glucose molecules which generate a return signal (containing decipherable glucose concentration information) that is picked up by a detector. This is not an invasive, test strip based testing device or method of measuring blood glucose concentrations, sometimes referred to as the “finger-stick” method. A wearable, minimally invasive glucose monitoring system is the “GlucoWatch G2 Biographer” system that was originally manufactured by Cygnus Inc. located in Redwood City, Calif. This device, likewise, does not utilize the well known test strip based technology of measuring blood glucose concentrations. Rather, this system extracts body fluids from the skin to estimate blood glucose concentrations. The system has well known drawbacks in that it is not as accurate as test strip based technologies, requires an expensive separate replaceable Autosensor (consumable), a two to three hour system warm-up period, and periodic calibration. Typical blood glucose readings in this system possess a 15 minute lag (the user's real-time blood glucose reading was actually the blood glucose concentration in the user's blood stream 15 minutes prior to the reading), compared to the finger-stick method that require mere seconds to get effectively a real-time blood stream (capillary) measurement. Additionally, reports of up to 50% of all users experience skin irritation.

The GenExel-Sein's DUO-CARE™ Combined Blood Glucose & Blood Pressure Monitor product is intended for home use, and provides a patient with two formerly separate monitoring devices in one convenient package. The unit is designed to be temporarily worn on the user's wrist during blood pressure measurements. The wrist band is actually a wide inflatable blood pressure cuff, and removed once the reading is obtained. This unit's is over 3.0 inches in length and weighs over 5.0 ounces (with batteries installed); such size and weight factors precludes such a device from inconspicuous, continuous wear. The unit contains a typical test strip based glucose monitoring system with the test strip portal located at the bottom of the unit. The location of the test strip portal would force awkward arm positioning if one attempts to wear the device while inserting the test strip into the unit. This maneuver becomes especially awkward if attempting to alternate the digits used for blood sampling. Furthermore, the device does not possess the means for storing any testing accessories such as test strips, calibration strips, lancets, and the like.

There remains a need for an all-in-one glucose testing/measuring device that is self-contained, portable and can be worn by a user throughout the day. The present invention fulfills this need and more.

### SUMMARY OF THE INVENTION

In accordance with a preferred embodiment of the present invention, there is disclosed a portable, glucose measuring device of such compact dimensions that it enables the device to be continuously worn. The device uses well known invasive electrochemical glucose test strip technology, and in other embodiments, possesses storage areas for blood glucose testing accessories such as glucose test strips, lances, and the like, defining an all-inclusive or an all-in-one type device. A fastening apparatus enables mounting of the device onto a user's limb, such as the wrist, forearm, upper arm, ankle, and the like.

Glucose measuring devices of adequate compact dimensions presently exist. Although the devices are not intended nor designed to be worn, their mere existence strongly contribute to enablement of the present invention. Examples of such compact glucose measuring devices are available from Home Diagnostics Inc. of Fort Lauderdale, Fla. Two compact products are available that are designed to mount onto small prescription medicine like bottles. The products are presently available under the monitoring product names SIDEKICK and TRUE2GO.

It is an object of the present invention to provide a portable glucose measuring or monitoring device that is compact, and continuously wearable. The glucose measuring device utilizes mature, well-known, invasive, test strip based glucose technologies used in many current blood glucose personal monitoring systems commercially available. Systems using such test strips are based on well known electrochemical methods of deciphering blood glucose concentrations by testing a capillary blood sample.

It is an object of the present invention to provide a wearable test strip based glucose monitoring device that is compact, all-inclusive such that the device includes storage areas for blood glucose accessories or necessary peripherals such as test strips, calibration strips, lancets, and the like.

It is an object of the present invention to provide a device that is sufficiently light, highly compact and portable, such that the monitoring device can be inconspicuously worn a user.

Whereas there may be many embodiments of the present invention, each embodiment may meet one or more of the foregoing recited objects in any combination. It is not intended that each embodiment will necessarily meet each objective. Thus, having broadly outlined the more important features of the present invention in order that the detailed description thereof may be better understood, and that the present contribution to the art may be better appreciated, there are, of course, additional features of the present invention that will be described herein and will form a part of the subject matter of this specification and claims.

In this respect, before explaining at least one embodiment of the invention in detail, it is to be understood that the invention is not limited in its application to the details of construction and the arrangements of the components set forth in the following description or illustrated in the drawings. The present invention is capable of other embodiments and of being practiced and carried out in various ways. For example, the embodiments shown and described are primarily intended for right-handed users where the device is typically worn on the left arm; however, the rearrangement of components to accommodate left handed users (where the device is worn on the right arm) is understood to be within the scope of the disclosed invention. Also, it is to be understood that the phraseology and terminology employed herein are for the purpose of description and should not be regarded as limiting.

### PARTICULAR ADVANTAGES OF THE INVENTION

The embodiment of a blood glucose measuring device in a wearable format, such as a wristwatch, provides a user or caregiver several advantages. Besides the convenience of the optional timekeeping function of the device, the wearable device encourages frequent testing by its mere presents. Additionally, the wearable (e.g. wrist mounted) device assists the user, or operator in the method or act of taking a blood glucose reading by freeing up the hand that would normally be handling the typical hand-held glucose meter. Furthermore, mounting the device on a user's limb will help prevent glucose meter mishaps and logistical issues in the testing procedure, including dropping, bumping, or contaminating the test strip, lancet, and the like. Locating a suitable clean surface to rest a typical hand-held meter and accessories on during hands washing, digit disinfecting, lance preparation and use, blood drop creation (digit squeezing), etc. can present a challenge, especially in uncooperative environments. Such challenges and more are overcome by the use of a wearable device. For example, the process of obtaining a blood glucose reading becomes much less conspicuous when compared to the familiar method of retrieving a typical glucose meter from a pocket or purse, and going through the blatantly revealing typical steps to obtain a reading. Again, the procedure to obtain a reading with a typical test strip based glucose meter often requires the use of a table, or the like, due to the sanitary methodology and the various components required in the process (components include: a blood glucose meter, test strips, a lance, lancing points, cleaning swabs and alcohol and the like).

The ability to start the testing process with a wrist mounted glucose meter with loaded test strip will free up the individual's hands, enabling them to focus on the next steps of the process in a more streamlined fashion. Such steps include: hands washing, lance manipulation, squeezing out blood sample, and navigating drop placement onto the end of the device mounted test strip. Streamlining the method of glucose monitoring should benefit many that require monitoring, especially those with active lifestyles.

It is well known in the glucose monitoring industry that the frequency of glucose monitoring by an individual is based on the fundamental three C's. The three C's are: Cost, Comfort and Convenience. Since the device can be easily and inconspicuously worn, an individual would be more willing to wear such a device, versus a bulky kit attached to one's belt or the like. There's a greater comfort level in social situations where no one would give such a worn device a second look, when the typical bulky glucose monitoring kit would tend to inadvertently draw attention. There isn't any expected change in cost per reading when compared to the typical test strip based hand-held monitoring systems. Additionally, the device provides a visual/tactile reminder which assists in maintaining the glucose monitoring regiment. It can be worn by either the individual who desires glucose monitoring or a caregiver involved in the monitoring process for the individual such as a health aid, parent, child guardian, etc.

### BRIEF DESCRIPTION OF THE DRAWINGS

The invention will be described by reference to the specification and the drawings, in which like numerals refer to like elements, and wherein:

FIG. 1 is a simple block diagram of a wearable, all-in-one, blood glucose monitoring system;

FIG. 2 is an orthogonal front view of one embodiment of a wearable, all-in-one, blood glucose monitoring system having automatic test strip and lancet dispensing systems;

FIG. 3A is an orthogonal front view of one embodiment of a glucose test strip modular/stack dispenser or cartridge, enabling automatic test strip dispensing;

FIG. 3B is an enlarged perspective front-side view of the glucose test strip exit port 44 of dispenser shown in FIG. 3A;

FIG. 4 is a cross-sectional side view of one embodiment of a lancet dispenser/actuation system;

FIG. 5 is a perspective top-side view of an embodiment of a wearable blood glucose monitoring system having a test strip carousel type receiver, enabling automatic test strip dispensing;

FIG. 6 is a perspective top-side view of an alternate embodiment of a wearable blood glucose monitoring system having a flip-top and a single compartment storage area;

FIG. 7 is a perspective top-side view of an alternate embodiment of a wearable blood glucose monitoring system having a flip-top and multi-compartment storage areas; and

FIG. 8 is a perspective top-side view of an alternate embodiment of a wearable blood glucose monitoring system having an unattached blood glucose monitoring portion designed to engage with a single compartment storage area.

The drawings are not to scale, if fact some aspects have been emphasized for a better illustration and understanding of the written description.

### PARTS LIST

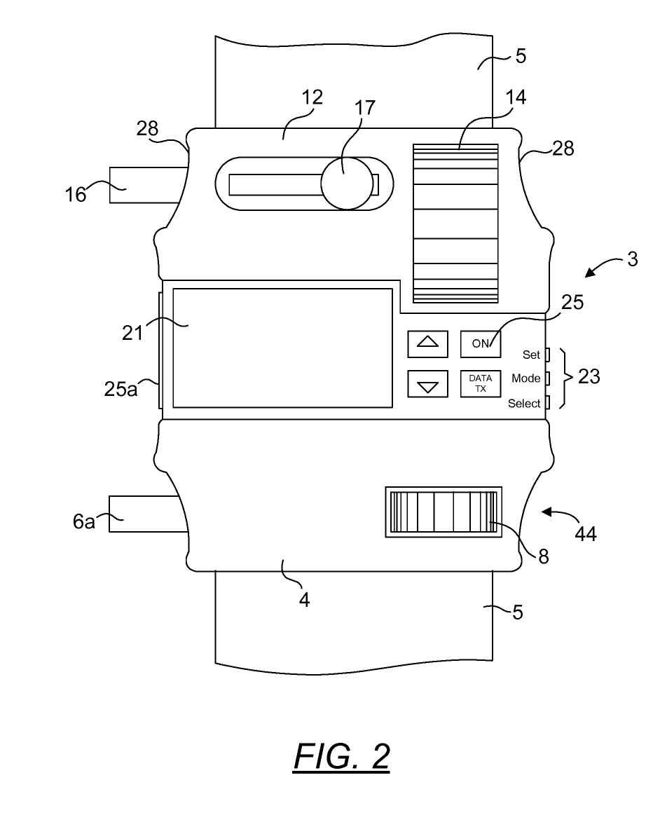

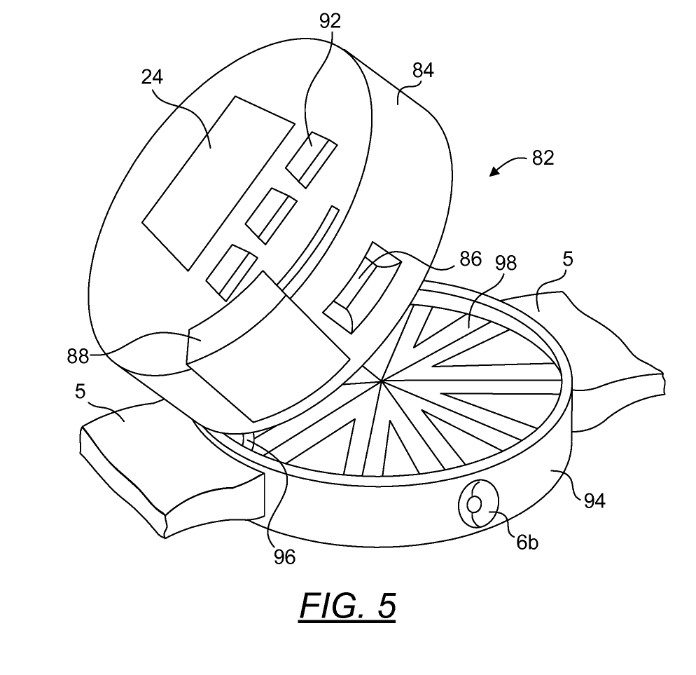

2. all-in-one blood glucose measuring system
3. wearable glucose measuring device
4. blood glucose dispenser analyzer system
4a. blood glucose dispenser device
5. straps
6a. test strip advance member (FIG. 2)
6b. test strip advance button (FIG. 5)
8. test strip ejector
10. glucose analyzer
12. lancet blood sampling system
12a. lancet dispenser/actuation device
14. drum lancet magazine
16. lancet actuator
18. lancet ejector
19. data processing subsystem
20. microprocessor
21. display
22. clock
23. clock function buttons
24. memory
25. glucose meter function keypad
25a. I/O (input/output) port
26. data interface
27. power supply
28. lancet output port
30. desiccant liner
32. leaf springs
34. coil spring
36. test strip plunger
38. active test strip
39. test strip contacts
40. test strips
41. test strip port connectors
42. exit tunnel
43. test strip housing
44. glucose test strip dispenser port
50. return springs
52. inner chamber
54. inner chamber spring connection point
56. outer chamber posts
58. spring
64. retaining posts
66. locking member
68. retaining spring
70. metal needle
72. strike plate
73. striking surface
74. active chamber
75. flexible retention clips
76. magazine axis
78. single storage unit walls
82. carousel style glucose meter wrist watch
84. compact blood glucose measuring device
86. test strip ejection port
88. selection cover
92. keypad
94. bottom watch housing
96. flip top hinge
98. carousel test strip receiver
104. glucose watch with single storage compartment
105. glucose watch with multiple storage compartments
106. sliding drawer inner compartment
107. sliding drawer outer cover
108. test strip manual port
109. housing dividing wall
110. subdivided compartment
111. wearable receiver assembly
116. housing (bottom storage compartment)
120. loose test strips
126. clock (stand alone)—for housing-strap assembly
128. USB memory/receptacle
130. mounting tabs
132. storage area

### DEFINITIONS OF THE TERMS USED IN THIS SPECIFICATION

The wearable monitoring system and all its embodiments thereof shall have equivalent nomenclature including: the device, the unit, the present invention, or the invention.

The term automatic shall mean a function provided mechanically such that a user does not have to directly handle a test strip or a lancet directly during testing. The term airtight shall further include the situation where at least one sacrificial test strip is exposed to the environment, thereby protecting the bulk of test strips remaining.

The term casual memory storage device shall mean an electronic storage device or component that is intended to be used as a common use flash drive, or the like, which is separate and distinct from any compact blood glucose measuring device present.

As it pertains to wearable device, the term compact shall mean a device (not including any limb fastening means) where no dimension exceeds 3.0 inches, and must not exceed 4.5 ounces in weight (including batteries).

The terms all-inclusive and all-in-one shall refer to wearable monitoring systems that provide a user with at least one storage area for at least one blood glucose accessory such as a test strip, lancet, and the like.

### DETAILED DESCRIPTION OF THE INVENTION

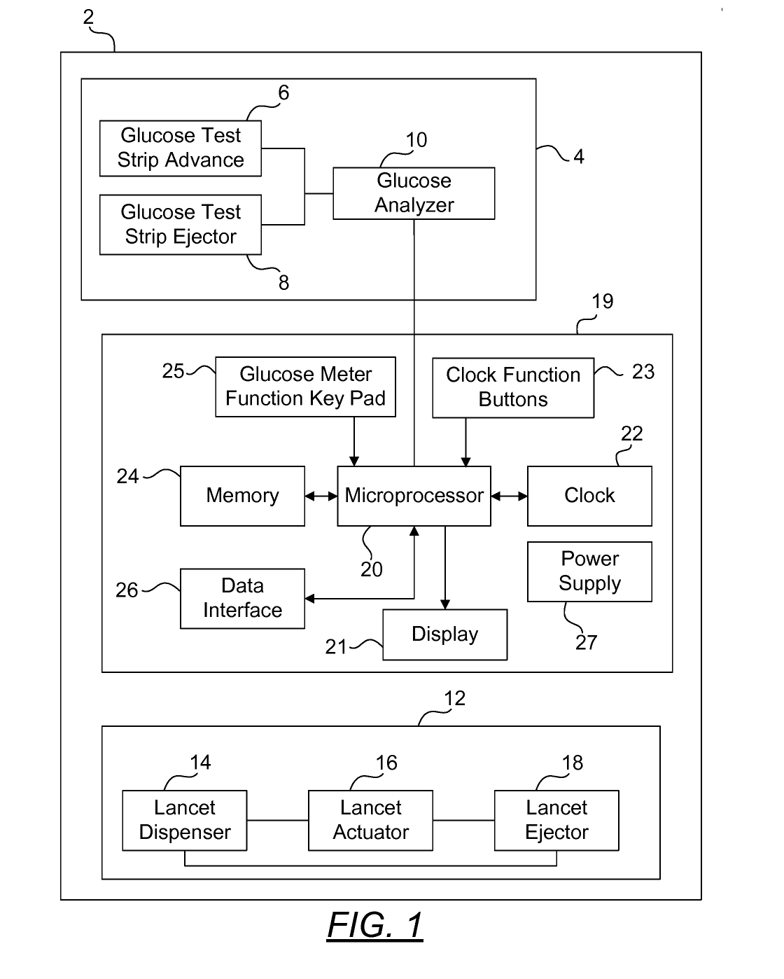

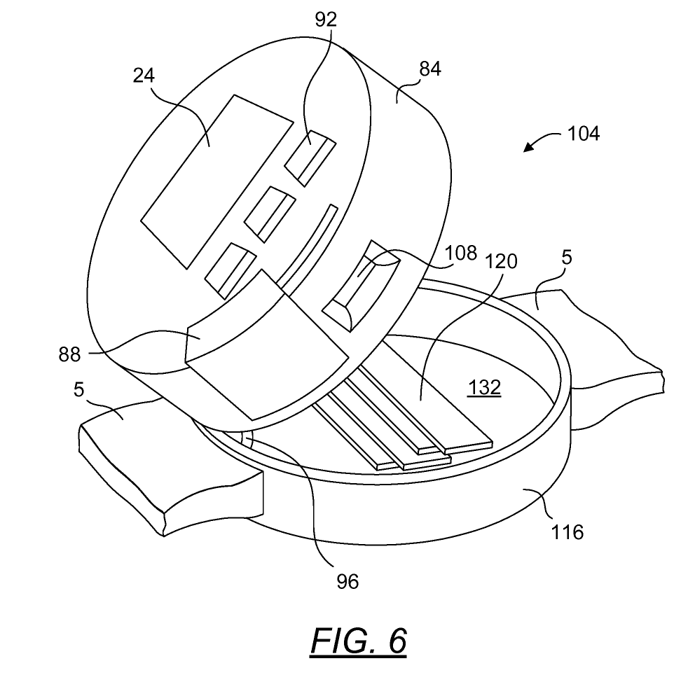

FIG. 1 is a block diagram for an all-in-one, blood glucose measuring system or device. In accordance with the present invention, and referring to FIG. 1, there is provided a block diagram of one embodiment of an all-in-one blood glucose measuring system 2. Disclosed are representative, novel, compact, wearable devices in a wristwatch type embodiment incorporating the all-in-one blood glucose measuring system 2, one such example is depicted in the embodiment of FIG. 2. The wristwatch format, however, is not intended as a structural limitation, but as one possible example. Integration and/or adaptation into commonly worn articles such as bracelets, armbands, wristbands, and the like, are also considered within the scope of the present invention. FIG. 2 shows an orthogonal front view of one embodiment of a wearable, all-in-one, blood glucose monitoring system having automatic test strip and an automatic lancet dispensing systems. Whereas, FIG. 5 depicts an embodiment of a wearable blood glucose monitoring system having a test strip carousel type receiver that enables automatic test strip dispensing sans a lancet dispensing system. FIGS. 6, 7 and 8, depict embodiments of a wearable blood glucose monitoring system having a portable blood glucose measuring device 84 both as a removable separate component as well as being hingedly connected to housing 116.

The glucose measuring technology incorporated is based upon the well known, mature, reliable, accurate, quick-response, non-continuous, test strip based measurement technologies which are sometimes referred to as episodic or intermittent glucose monitoring technologies. Test strip based glucose monitoring systems are considered invasive, i.e. systems which require a capillary blood sample to estimate the individual's blood glucose concentration. Such samples are normally obtained by lancing a finger tip or an approved alternate test site to obtain such a capillary blood sample. More recent methods of producing a blood sample involve the use of a laser (FDA-cleared LASETTE® by Cell Robotics International Inc.), a device that burns a small opening to the blood carrying sub-layers of the skin.

Additionally, test strip based meter measurement systems under discussion can be further classified as the second generation glucose monitoring type systems.

These systems are characterized by an electrochemical measurement based upon a reaction with blood glucose that generates an electrical current, when read by corresponding electronics, whose magnitude corresponds to the blood glucose concentration of the test sample. In such a system, one only needs to supply an adequate blood sample to a ready meter (test strip inserted, calibrated unit) and wait for a reading to appear on the display. The first generation glucose monitoring type was based upon photometric type measurements (color changes), and was plagued by required test strip blotting, wiping, manual timing, in addition to difficult to decipher color shifts.

It is expected that those with implantable sensors, where frequent sensor recalibration requires the use of an invasive test strip monitoring system (mainly due to protein contamination of the implant), would welcome such a complementary convenient device. It can also serve as the recommended emergency backup monitoring device to confirm a hypoglycemic reading. Hypoglycemic false alarms are common and can be caused by movement that causes the sensor to be cut off or restricted from the interstitial fluids being sampled.

Referring to FIGS. 1, 2 and 5, a test strip is automatically positioned into the glucose analyzer 10 of FIG. 1 by cycling the glucose test strip advance member 6a and 6b (in embodiments depicted in FIGS. 2 and 5) or via manual placement of a test strip into the glucose analyzer 10 of FIG. 1 (in the embodiment depicted in FIGS. 6, 7, and 8). When a test strip is properly positioned in glucose analyzer 10 of FIG. 1, the blood glucose dispenser analyzer system 4 disables the signal from clock 22 from being posted on display 21 via microprocessor control 20. It is understood that clock 22 is an optional component that is not absolutely necessary when taking blood glucose measurements. In addition, the blood glucose dispenser analyzer system 4 sends the blood glucose analysis data, when ready, to microprocessor 20, which forwards the results to display 21 for viewing. The manual removal of the test strip or the mechanized removal by the glucose test strip ejector 8 from glucose analyzer 10 will flag microprocessor 20 of data processing subsystem 19 to return control of display 21 back to clock 22 such that the date/time and the like can be displayed. The used lancet can optionally be ejected and disposed of using the lancet ejector 18 at this time.

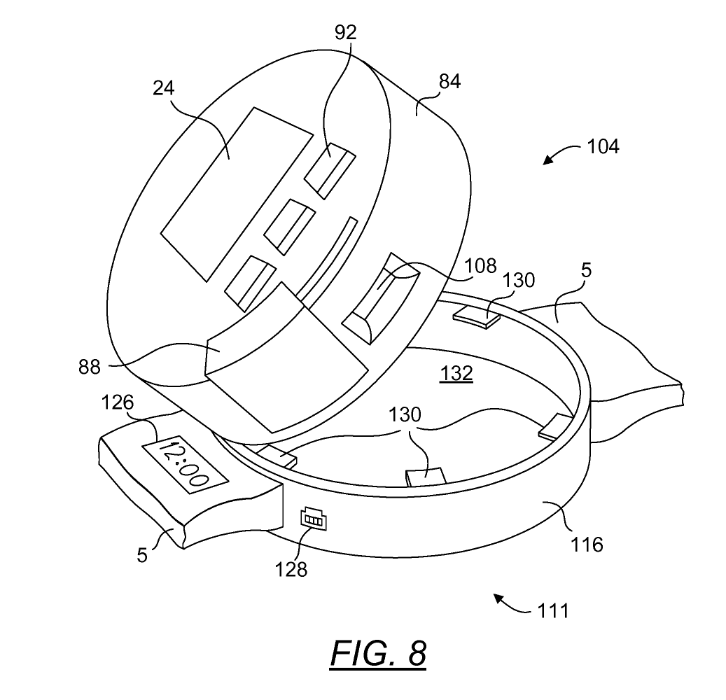

Again referring back the block diagram of FIG. 1, the optional, although highly useful clock 22 should provide at a minimum, date and time information, not only for viewing on display 21, but also to provide microprocessor 20 with a date and time stamp associated with each glucose measurement taken by the blood glucose dispenser analyzer system 4, for storage in memory located in memory 24 for later retrieval and/or analysis. Clock 126 depicted in FIG. 8 is available for portable blood glucose measuring device 84 not possessing an integrated clock, or can act as a secondary standalone timepiece separate from portable blood glucose measuring device 84. Such standalone timepiece type units are well known and commonplace.

The date and time values on clock 22 can be set via clock function buttons 23, which typically possess the well known, typical adjustment buttons entitled “SET”, “MODE”, and “SELECT”. Clock 22 adjustments can also be set via microprocessor 20 by obtaining the correct values from an external computer (not shown) delivered by data interface 26. In one embodiment, data interface 26 is a wireless device, enabling wireless data transmission/reception from an external device. Other data interface links include IR LED and USB which are all well known and commonplace technologies. In preferred embodiments, clock 22 contains programmable alarm features such as audible and/or vibratory, to assist the wearer in proper glucose monitoring and is of the quartz digital type. Other embodiments include the use of an analog type timepieces, such designs give the device a particular type of design appearance that may appeal to some users. In situations, where a non-digital or analog clock is used, clock function will be appropriated by a digital internal clock, incorporated into, or attached to microprocessor 20. Such a digital internal clock will provide the desired date/time stamp with all glucose measurements.

Again, referring back to block diagram of FIG. 1, memory 24 contains both ROM and RAM type memories. One example of ROM memory can is the EPROM programmable type. RAM memory will preferably be of the static type, where memory contents remain intact during power supply interruptions, for example, when changing batteries. The ROM portion of memory will be primarily dedicated to the blood glucose dispenser analyzer system 4, where memory functions include data required for deciphering electrical characteristics of active test strips, such as calibration data and the like. Such information is required in the conversion of raw data into a blood glucose concentration value. A permanent electronic lookup table residing in ROM memory is one method that will provide translational information to microprocessor 20 enabling glucose analyses. RAM memory will provide temporary storage for all the glucose readings and associated date/time stamp information. In preferred embodiments, the RAM memory will have enough capacity to hold at least one month's worth of readings. This would require a memory capacity capable of storing a least 180 readings, typical for an individual that monitors six times a day.

Again, referring back to block diagram of FIG. 1, microprocessor 20 is programmed to primarily analyze the information provided by the blood glucose dispenser analyzer system 4 to calculate blood glucose concentration values. In preferred embodiments, microprocessor 20 contained in wearable glucose measuring device 3, is connected to data interface subsystem 26 where bilateral communications from an external computer, PDA, and the like can be initiated. Data interface subsystem 26 includes an input/output means controlled by microprocessor 20 and glucose meter function keypad 25 to manage the data stored in RAM memory 24. For example data stored in memory 24 can be copied to an external device, or information such as date/time information, alarm settings, and the like, can be transferred to a wearable glucose measuring device. Well known interfacing technologies used for such communications include those based on: IR-Infrared, RF-Radio Frequency; as well as hard wired technologies such as USB, Firewire-1394, and the like.

Again, referring back to block diagram of FIG. 1, all-in-one blood glucose measuring system 2 is energized by power supply 27. Power supply 27 includes portable power sources such as batteries. More particularly, watch type batteries; that are available in various forms such as alkaline, lithium, silver oxide, and rechargeable versions such as Li-ion and the like.

Lancet blood sampling system 12 of FIG. 1 includes, as depicted in preferred embodiment FIG. 2, the functional integration of the lancet dispenser 14, lancet actuator 16, and the lancet ejector. All such functions are all incorporated into wearable glucose measuring device 3 depicted in FIG. 2. The function of the lancet blood sampling system 12 is to furnish the user the means for delivering a blood sample, of adequate volume, to the receiving end of a prepared glucose test strip loaded in glucose analyzer 10. The alternate embodiment of glucose meter wrist watch 82, depicted in FIG. 5, does not possess the built-in lancet apparatus. Therefore, the lancing function associated with this embodiment must be accomplished by a separate device or apparatus, such as a lancing device.

Such separate lancing systems are well known and commonly available. Lancing systems typically include the consumable lancet, sharps, or points, that mount into a lancing or lancet device; such devices help automate the lancing process. The embodiment of FIG. 6, depicting glucose watch with single storage compartment 104, is capable of storing a small quantity of simple lancets (not shown) and loose test strips 120, in storage area 132 located at the bottom of housing 116. It is understood that the contents of storage area 132 can vary to suit the preferences of the user.

FIG. 2 depicts a preferred embodiment of wearable glucose measuring device 3 that contains both lancet system 12 and blood glucose dispenser/analyzer system 4 incorporated in fixed, predetermined locations. Wearable glucose measuring device 3 is fastened to a user's wrist, or the like, by a fastening means, such as bands or straps 5. Such bands or straps 5 can be constructed from either the one or two pieces of fastening material. Typical characteristics of preferred fastening materials include those that are sufficiently flexible, durable material such as leather, rubber, fabric, and the like. Other fastening means include the use of rigid materials such as when a bracelet-like appearance is desired; common materials used in such a situations include metals, ceramics, and hard plastics. As will be apparent, other means of attaching wearable glucose measuring device 3 to a user may be suitably adapted for use with the present invention without departing from the spirit thereof.

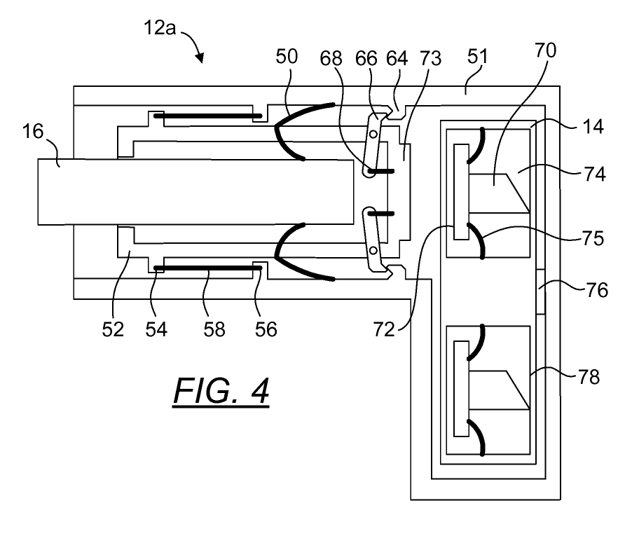

FIG. 4 is a cross-sectional side view of one possible embodiment of a lancet blood sampling system 12 depicted as lancet dispenser/actuation device 12a, incorporated into the device of FIG. 2. Lancet dispenser/actuation device 12a of FIG. 4 includes the following: lancet actuator 16, cocking knob 17 (shown in FIG. 2), replaceable drum lancet magazine 14, and lancet output port 28 (shown in FIG. 2). Replaceable drum lancet magazine 14 enables lancet replenishment in batch format. User loads replaceable drum lancet magazine 14 into lancet dispenser/actuation device 12a. Drum lancet magazine 14 is then rotated about magazine axis 76 (depicted in FIG. 4) into position where linear alignment is achieved between actuator 16, active chamber 74 and strike plate 72, both depicted in FIG. 4. Audible and tactile detent positions assist in the positioning task of drum lancet magazine 14. Cocking knob 17 (depicted in FIG. 2) is slid left toward the lancet actuator 16 member until locked into position. A prepared finger tip, or the like, of the free limb or arm, not possessing wearable glucose measuring device 3, is placed against lancet output port 28 while lancet actuator 16 is depressed by the thumb of the same hand. Depressing lancet actuator 16 releases inner chamber 52 propelling it toward strike plate 72, where a quick, high impact force is transferred to metal needle 70, whereby resultant motion lances the awaiting fingertip urged against lancet output port 28. The user then proceeds to place the blood sample on the test strip loaded in test strip port 44 of FIG. 2.

The following discussion will address additional system details of lancet dispenser/actuation device 12a depicted in FIG. 4, which provides one embodiment of lancet blood sampling system 12 shown in block diagram form in FIG. 1. Referring to FIGS. 2 and 4, cocking knob 17 is connected to inner chamber 52 and is slid left toward lancet actuator 16 member until locked into position by locking member 66 locking onto the two outer chamber posts 56, placing spring 58 in tension. One end of spring 58 is connected to inner chamber 52 at the inner chamber spring connection point 54, while the opposite end is connected to outer chamber posts 56. Lancet actuator 16 position is retained by return springs 50. When depressing lancet actuator 16, retaining spring 68 is temporarily compressed releasing locking member 66 from retaining posts 64 causing inner chamber 52 to impact metal needle 70 strike plate 72 with striking surface 73. Flexible retention clips 75 are attached to the lancet single storage unit walls 78, and hold the metal needle components or lancets in proper alignment during use and in storage.

Referring to FIG. 2, display 21 is an LCD type display, commonly found in low power portable devices such as wrist watches. Other timepiece displays may be of the analog type (not shown) to give the device a particular type of look or appearance, in such a situation, a separate digital type display (e.g. LCD, LED) is necessary to display glucose blood concentrations, review past glucose information, and the like. Clock function buttons 23 are used to adjust the date, time, and alarm settings of the digital LCD clock system whose output is posted on display 21. Such clock function buttons 23 are typically entitled “SET”, “MODE”, “SELECT”, and the like; and their method of use is well known. The I/O (input/output) port 25a uses common communications type technologies such as IR-Infrared, RF-Radio Frequency, and hard-wired technologies such as USB, Firewire-1394, and the like.

Referring to FIG. 2, glucose meter function keypad 25 is a multifunctional keypad whose functions include: stepping through glucose data stored in memory, viewable on display 21, activating the I/O (input/output) port 25a, initiating a communication link, toggle between clock mode and glucose meter modes, and the like.

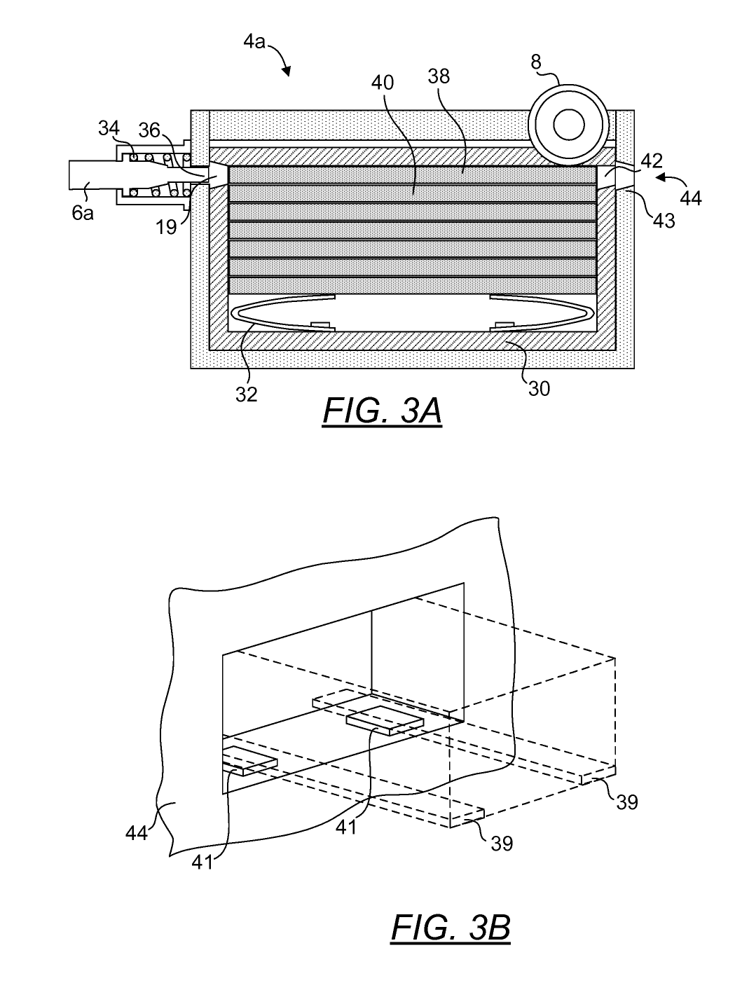

FIG. 3A is an orthogonal front view of one embodiment of a glucose test strip modular/stack dispenser or cartridge, enabling automatic test strip dispensing as well as replenishment of glucose test strips into the device in a batch format. FIG. 3B is an enlarged perspective front-side view of the glucose test strip dispenser port 44 of FIG. 3A. Referring to FIG. 1, blood glucose dispenser analyzer system 4 and subsystems are depicted in FIGS. 3A and 3B which are system details of the device of FIG. 2. Test strip advance member 6a is a normally extended push button mechanism that is biased away from active test strip 38 by coil spring 34. During a testing session, a user depresses test strip advance member 6a button toward active test strip 38, thereby causing test strip plunger 36 to contact one end of active test strip 38, moving the blood sample test end of the active test strip 38 through exit tunnel 42 of glucose test strip dispenser port 44 preparing the test strip for receiving the blood sample. When test strip advance member 6a is fully depressed, an electrical connection is made from the test strip port connectors 41 and the test strip contacts 39 residing on active test strip 38 (see FIG. 3B). Test strip port connectors 41 electrically connect the active test strip 38 to glucose analyzer 10 shown in FIG. 1.

In FIG. 3A, once active test strip 38 has been used, rotating the wheel-based test strip ejector 8 counterclockwise will eject used active test strip 38 out of glucose test strip dispenser port 44. Test strips 40 remaining supply, in preferred embodiments, is tracked by the microprocessor 20 of FIG. 1. Useful information such as test strips remaining, or an out-of-test-strips alarm, or the like, is displayed on display 21 of FIG. 1. Once a test strip is ejected from blood glucose dispenser device 4a, leaf springs 32 will lift the remaining stored test strips 40 upward, creating a new active test strip 38 and resetting the testing cycle to process additional readings. Desiccant liner 30 covers a portion of the inner surface of the test strip housing 43 of blood glucose dispenser device 4a. The function of desiccant liner 30 is to decrease water vapor concentration, thereby extending the shelf life of test strips 38 and 40.

FIG. 5 depicts an embodiment of a carousel style glucose meter wrist watch 82. Carousel test strip receiver 98 is designed to accommodate a carousel style test strips (not shown), or to accept individual test strips arranged in a circular fashion. A reference depicting a replaceable, ten test strip cartridge is described in U.S. Pat. No. 5,575,403, entitled “DISPENSING INSTRUMENT FOR FLUID MONITORING SENSORS”. A carousel style test strip cartridge or individual test strips are loaded in FIG. 5 by lifting compact blood glucose measuring device 84, which also functions as a lid to bottom watch housing 94, and placing the carousel style test strip cartridge onto the test strip carousel receiver 98. Compact blood glucose measuring device 84 is connected to the bottom watch housing 94 by flip top hinge 96. The bottom watch housing 94 is connected to bands or straps 5 which attaches to a user's wrist. Such a band or straps 5 can be of either a one or two piece design, and is constructed of a sufficiently flexible, durable material such as leather, rubber, and the like. Compact blood glucose measuring device 84 in preferred embodiments, mates tightly with bottom watch housing 94 providing an airtight and/or water proof seal to help protect the internal workings and components of device 82 from dirt, moisture, and the like. When depressed, test strip advance button 6b cycles the test strip carousel and advances a test strip through test strip ejection port 86. The features and function of test strip ejection port 86 is similar in scope to the operation of test strip exit port 44 of FIG. 3B.

Referring to FIG. 5, compact blood glucose measuring device 84, keypad 92 possesses all the features and functions to both shown and described elements 25 and 21 in FIG. 2. Display 24 of FIG. 5 is fully described by the features, function, and description associated with display 21 of FIG. 2. Selection cover 88 is a sliding cover that allows access to either test strip ejection port 86 or I/O port (not shown), but not both simultaneously; this mechanical feature provides an additional level of safety for the user. Selection cover 88 helps prevent the undesirable situation where a hard wired technology such as USB, Firewire-1394 type cable is connected to the I/O port, while user is attempting to take a blood glucose reading via mounted test strip in test strip ejection port 86. Even though there are many electrical isolation type safe guards available for such situations, this additional feature will enhance the level of patient safety both actual and perceived. An alternate embodiment (not shown) is accomplished by using the connector of a cable associated with a hard wired technology such as USB, Firewire-1394, and the like to simultaneously connect to the device of FIG. 5 while physically blocking the test strip ejection port 86 such that a test strip can not be inserted nor dispensed.

Referring to FIG. 5, compact blood glucose measuring device 84, would benefit from a protective-clear cover (not shown) that can be used to provide an airtight and/or water proof seal to protect the elements located on the top watch housing 84 from dirt, moisture, and the like. Such a protective-clear cover (not shown) can be removably attached or in the form of a flip-top. Additionally, gaskets, O-rings, and the like can be used to provide sealing properties.

The glucose watch with single storage compartment 104 of FIG. 6 is yet another embodiment of an all-in-one glucose meter wrist watch. Compact blood glucose measuring device 84, which also acts as a lid, is connected to bottom storage compartment 116 via flip-top hinge 96. The bottom storage compartment 116 is connected to bands or straps 5 which attaches to a user's wrist. Such a band or straps 5 can be of either a one or two piece design, and is constructed of a sufficiently flexible, durable material such as leather, rubber, and the like. In a preferred embodiment the top watch housing 84 mates tightly with housing (bottom storage compartment) 116 providing an airtight and/or water proof seal to help protect the items stored in storage area 132 from dirt, moisture, and the like. In this embodiment loose test strips 120, small lancets (not shown), are conveniently stored in storage area 132 and must be individually removed and manually manipulated by the user.

Referring to FIG. 6, compact blood glucose measuring device 84, keypad 92 possesses all the features and functions to both shown and described elements 25 and 21 of FIG. 2. Display 24 of FIG. 6 is fully described by the features, function, and description associated with display 21 element in FIG. 2. Selection cover 88 is a sliding cover that allows access to either test strip ejection port 86 or I/O port (not shown), but not both simultaneously; this mechanical feature provides an additional level of safety for the user. Selection cover 88 helps prevent the undesirable situation where a hard wired technology such as USB, Firewire-1394 type cable is connected to the I/O port, while user is attempting to take a blood glucose reading via mounted test strip in test strip manual port 108. Even though there are many electrical isolation type safe guards available for such situations, this additional feature will enhance the level of patient safety both actual and perceived. An alternate embodiment (not shown) is accomplished by using the connector of a cable associated with a hard wired technology such as USB, Firewire-1394, and the like to simultaneously connect to the glucose watch with single storage compartment 104 while physically blocking test strip manual port 108 such that a test strip cannot be inserted nor dispensed.

Referring to FIG. 6, compact blood glucose measuring device 84, a protective-clear cover (not shown) can be used to provide an airtight and/or water proof seal to protect the elements located on compact blood glucose measuring device 84 from dirt, moisture, and the like. Such a protective-clear cover (not shown) can be removably attached or in the form of a flip-top embodiment which is attached to a hinge on one end and utilize gaskets, O-rings, and the like to provide the sealing properties.

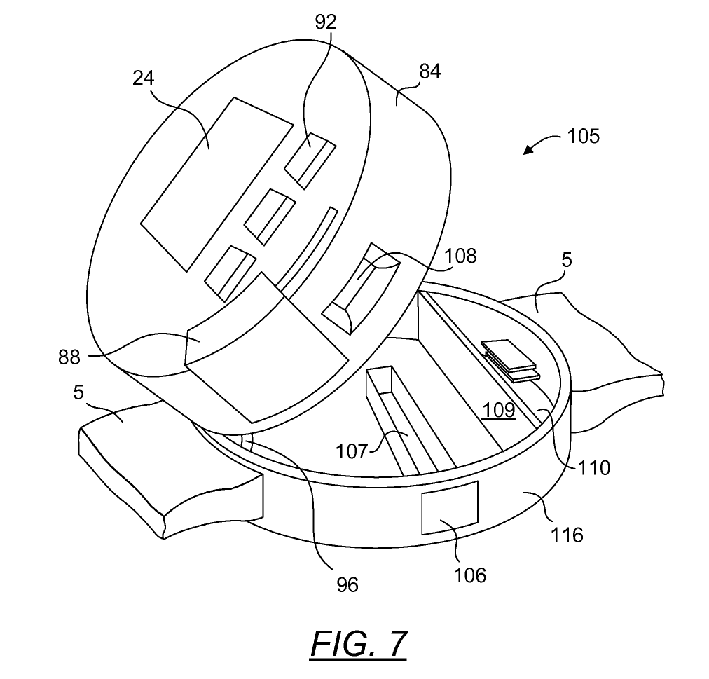

FIG. 7 is a perspective top-side view of an alternate embodiment of a wearable blood glucose monitoring system having a flip-top, multi-compartment storage area. Glucose watch with multiple storage compartments 105 is depicted in FIG. 7. The glucose watch with multiple storage compartments 105 differs from glucose watch with single storage compartment 104 of FIG. 6 only with respect to the number and type of storage compartments presented. Device 105 depicts a slide-out drawer compartment comprising a sliding drawer inner compartment 106 and an attached sliding drawer outer cover 107. Another storage option is created by housing dividing wall 109, where single storage area 132 of FIG. 6 is subdivided into smaller storage areas. Such a storage compartment schemes and the like, present a variety storage options that provide the user with additional benefits. Benefits include increased organizational capability, and ease of locating and obtaining specific accessories such as test strips, lancets, and the like. Additionally, isolated storage areas can be used to store used accessories such as test strips, lancets, without fear of contaminating unused accessories.

FIG. 8 is a perspective top-side view of an alternate embodiment of a wearable blood glucose monitoring system having an unattached blood glucose monitoring portion designed to engage with a compartment storage area. Wearable receiver assembly 111 incorporates a removable attaching means. One example of such an attaching means is accomplished using mounting tabs 130. Mounting tabs 130 are designed to cooperate with unattached compact blood glucose measuring device 84 such that the unit is manually secured over storage area 132. Wearable receiver assembly 111 is designed to possess a variety of features or options to assist the user in their daily activities as well as in the blood glucose monitoring process. One such feature is clock 126; Clock 126 is a standalone timepiece separate from the clock that may be present on the mating compact blood glucose measuring device 84. The primary function of clock 126 is to act as a typical ordinary timekeeping device. It is understood that such ordinary timekeeping devices can include alarms, hourly chimes, reminders, and the like.

Another useful feature is the USB memory/receptacle 128 intended to function as a casual memory storage device. USB memory/receptacle 128 is comprised of a memory circuit and an I/O circuit. The user would use USB memory/receptacle 128 as one would use an ordinary USB flash drive device, or the like. USB flash drive devices are well known and commonplace, and are used to store a variety of information. It is understood that the receptacle portion of USB memory/receptacle 128 will likely have a smaller USB connector format in cooperation with the size of the device such as Mini-b, Micro-AB, Micro-B, and the like. A conversion device or cord is necessary to enable attachment into a common USB series “A” receptacle.

As such, those skilled in the art will appreciate that the conception, upon which this disclosure is based, may readily be utilized as a basis for the designing of other structures, methods and systems for carrying out the several purposes of the present invention. It is important, therefore, that the invention be regarded as including such equivalent construction insofar as they do not depart from the spirit and scope of the conception regarded as the present invention.

What is claimed herein is:

1. A continuously wearable device adapted to receive a compact blood glucose measuring device comprising a housing that accommodates a compact blood glucose measuring device using invasive, electrochemical, single use glucose test strip based technology, wherein the housing is adapted to receive a fastening means that cooperates with the housing to provide a means for mounting the continuously wearable device on a limb.

2. The continuously wearable device of claim 1, wherein said continuously wearable device further comprises a storage means, whereby said storage means provides a holding area for blood glucose accessories.

3. The continuously wearable device of claim 2, wherein said storage means is substantially airtight.

4. The continuously wearable device of claim 1, wherein said continuously wearable device further comprises a clock.

5. The continuously wearable device of claim 2, wherein said storage means is comprised of at least two discrete storage areas.

6. The continuously wearable device of claim 1, wherein said continuously wearable device further comprises a casual memory storage device that cooperates with USB readers.

7. The continuously wearable device of claim 1, wherein said compact blood glucose measuring device is removably attachable to the continuously wearable device.

8. A continuously wearable compact blood glucose measuring device comprising a housing containing a compact blood glucose measuring device using invasive, electrochemical, single use glucose test strip based technology disposed therein, wherein said housing is adapted to receive a fastening means, whereby said fastening means, in cooperation with said housing, provides a means for mounting said continuously wearable device on a limb.

9. The continuously wearable compact blood glucose measuring device of claim 8, wherein said housing further comprises a storage means whereby said storage means provides a holding area for blood glucose accessories.

10. The continuously wearable compact blood glucose measuring device of claim 9, wherein said storage means is substantially airtight.

11. The continuously wearable compact blood glucose measuring device of claim 8, wherein said continuously wearable compact blood glucose measuring device further comprises a clock.

12. A continuously wearable compact blood glucose measuring device comprising a housing having a compact blood glucose measuring device using invasive, electrochemical, single use glucose test strip based technology disposed therein, wherein said housing is adapted to receive a fastening means, whereby said fastening means, in cooperation with said housing, provides a means for mounting said continuously wearable device on a limb and wherein said housing, in cooperation with said compact blood glucose measuring device, has a means for automatically engaging a glucose test strip such that said glucose test strip is ready to receive a blood sample.

13. The continuously wearable compact blood glucose measuring device of claim 12, wherein said housing further comprises a storage area for storing a plurality of glucose test strips such that automatic dispensing of the glucose test strips is enabled.

14. The continuously wearable compact blood glucose measuring device of claim 13, wherein said plurality of glucose test strips is contained in a cartridge format, whereby replenishment of the glucose test strips into said continuously wearable compact blood glucose measuring device is accomplished in a batch format.

15. The continuously wearable compact blood glucose measuring device of claim 13, wherein said storage area is substantially airtight.

16. The continuously wearable compact blood glucose measuring device of claim 12, wherein said housing further comprises a means for automatically engaging a lancet such that said lancet is prepared to draw a blood sample.

17. The continuously wearable compact blood glucose measuring device of claim 16, wherein said housing further comprises a storage area for storing a plurality of lancets such that automatic dispensing of the lancets is enabled.

18. The continuously wearable compact blood glucose measuring device of claim 17, wherein said plurality of lancets is contained in a cartridge format, whereby replenishment of said plurality of lancets into the continuously wearable compact blood glucose measuring device is accomplished in a batch format.

19. The continuously wearable compact blood glucose measuring device of claim 12, wherein said continuously wearable compact blood glucose measuring device further comprises a clock.

20. The continuously wearable compact blood glucose measuring device of claim 12, wherein said continuously wearable device further comprises a casual memory storage device that cooperates with USB readers.
